(19) United States
(12) Patent Application Publication
(10) Pub. No.: US 2019/0022274 A1
(43) Pub. Date: Jan. 24, 2019

(54) FORMATION OF SEMI-PERMEABLE POROUS ARTIFICIAL SCAB

(71) Applicants: Parsival VEGA VILLAVICENCIO, Buenos Aires (AR); Miguel Edgardo BATTISTA, Buenos Aires (AR)

(72) Inventors: Parsival VEGA VILLAVICENCIO, Buenos Aires (AR); Miguel Edgardo BATTISTA, Buenos Aires (AR)

(21) Appl. No.: 16/115,874

(22) Filed: Aug. 29, 2018

Related U.S. Application Data
(63) Continuation of application No. 14/655,677, filed on Sep. 21, 2015.

(30) Foreign Application Priority Data
Dec. 27, 2012 (AR) 2012 0105043

(51) Int. Cl. A61L 26/00; A61K 31/164; A61K 31/185; A61K 31/573; A61K 31/655; A61K 31/136; A61K 31/7036

(52) U.S. Cl. CPC A61L 26/0085; A61L 26/0023; A61K 31/164; A61K 31/185; A61K 31/573; A61K 31/655; A61L 2300/442; A61K 31/136; A61K 31/7036; A61L 2300/222; A61L 2300/406; A61L 2300/412; A61L 2300/43; A61L 26/0066

(57) ABSTRACT

Porous semi-permeable artificial self-attaching scab designed to protect the eroded or injured surfaces. It can be applied on any living being (including plants). The polymerized mesh of the scab is formed when applied on the injured area by a process in which the lysed cells release enzymes that catalyze the reaction. Under this protective surface, the growth of new normal cells that can breathe freely without drying up is promoted. As a result, loss of water, proteins and electrolytes can be avoided; infections can be prevented; and the use of dressing and gauze bandage can be eliminated since they harm the new skin removed. The aqueous thixotropic red-colored fluid applied by brushing or spraying was obtained as a result of the reaction occurred in the container by the combination of the following substances: azosulfamide 1% to 5%+gentian violet 0.004% to 0.007%+dexamethasone 0.001% to 0.003′%+pantothenyl alcohol 0.5% to 2%+gentamicin 0.03% to 0.06%.

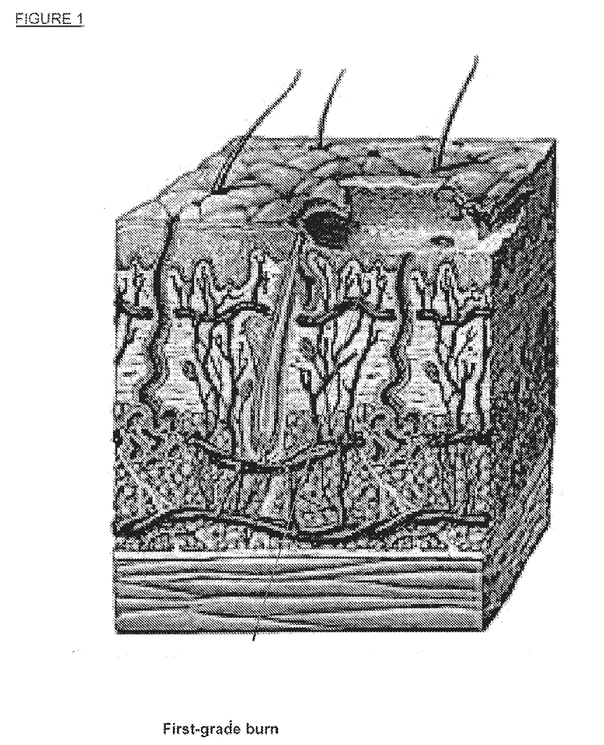

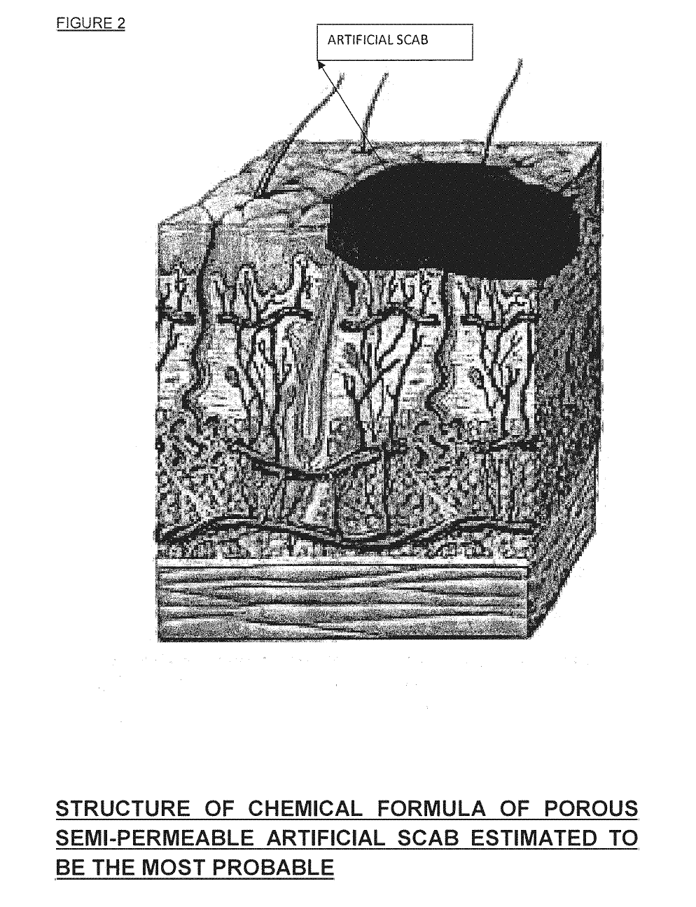

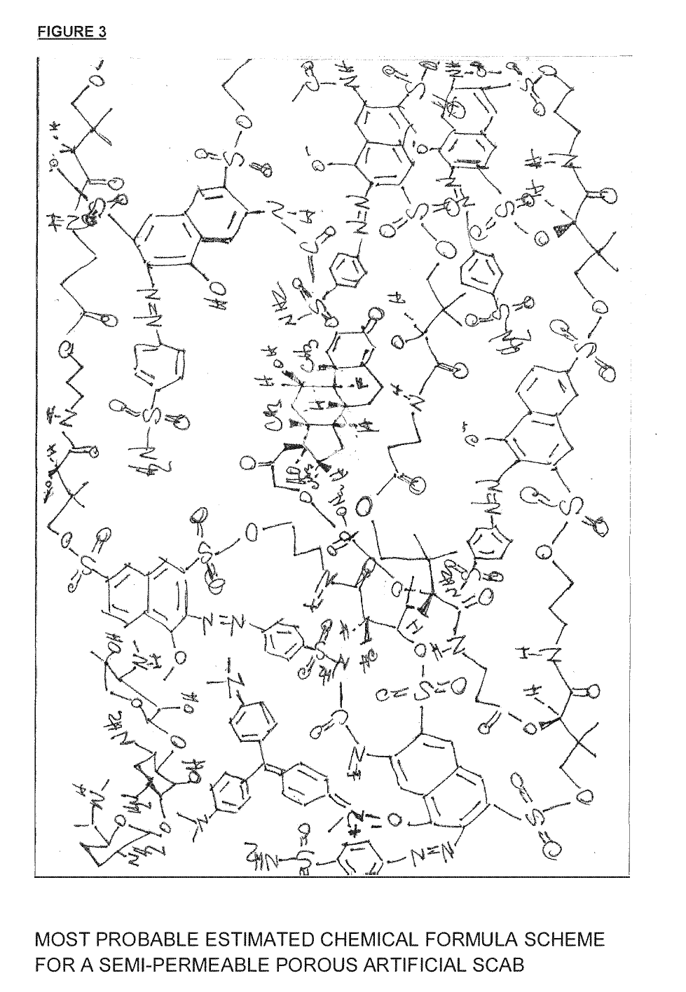

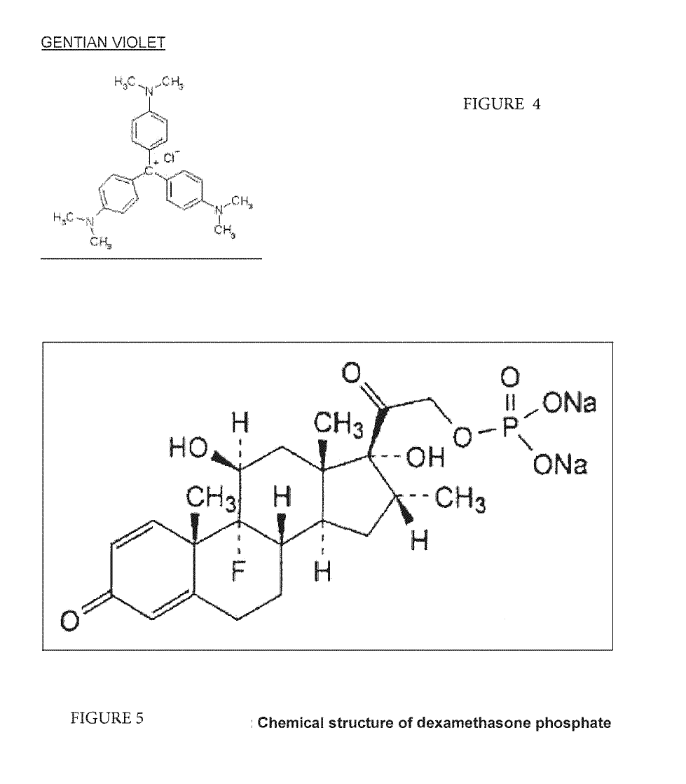

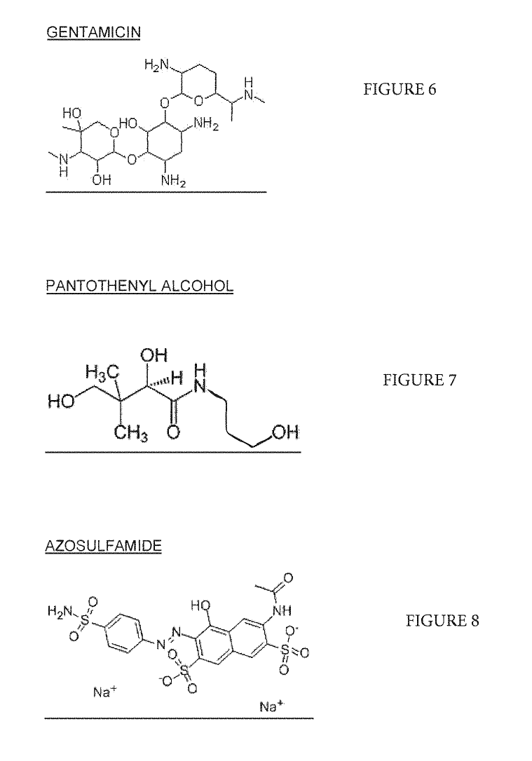

### CROSS-REFERENCE TO RELATED APPLICATIONS

This application is a continuation of pending U.S. patent application Ser. No. 14/655,677, filed Sep. 21, 2015 and titled “FORMATION OF SEMI-PERMEABLE POROUS ARTIFICIAL SCAB”, and the National Phase application of International Application No. PCT/PE2013/000010, filed Nov. 4, 2013, which further claims the benefit of foreign priority to Argentine No. 2012 0105043, filed Dec. 27, 2012, the disclosure of which are incorporated herein in their entireties.

### 1. FIELD OF THE INVENTION

The present invention relates to artificial cicatrization means by means of which a substance is applied to the damaged surfaces, wounds, burns A-AB, after removing the necrotic tissue and disinfecting the damaged or burnt zone.

### 2. PRIOR ART AND PROBLEMS TO BE SOLVED

The technique consists of the removal of the necrotic tissue from the injured zone and subsequent application of the artificial covering mentioned in the title. The preparation causes the formation of an artificial scab on the injured surface.

The problems to be solved are:

1. To replace the use of gauzes since they adhere to the damaged zone and cause lesions in the granulation zone when an attempt is made to remove them, thus causing defective cicatrizations.

2. To avoid potential infections given that protection against external biological agents is provided.

3. To avoid the loss of body fluids and heat, resulting from the loss of the integrity of the mechanical barrier.

The porous artificial scab allows the formation of new tissue, allowing its natural respiration: during its regeneration and bringing about a faster development of cicatrization by avoiding disrupted processes such as the necrosis or the development of anaerobes, without the need to cover and without performing further treatments until the natural detachment of the “Scab.”

### 3. AIM OF THE INVENTION

To protect the lesion against infections and losses of fluids with an artificial scab, and thus the cicatrization is much more rapid and the new tissue is not defective.
The usual cost of materials decreases, since once the scab is formed, further treatments are not necessary and the expulsion of this scab is natural.

### 4. DETAILED DESCRIPTION OF THE INVENTION

This aqueous thixotropic fluid which forms due to the reaction in the container is produced by the combination of 5 substances; azosulfamide between 1 and 5%+gentian violet between 0.004 and 0.007%+dexamethasone between 0.001 and 0.003%+panthenol between 0.5 and 2%+gentamycin between 0.03 and 0.06% having a red color. The preparation keeps its original properties for two years kept in caramel colored glass closed by screw cap or in pressure plastic at ambient temperature between 12 and 15 degrees centigrade.

In the case of extensive lesions, this red colored liquid is eliminated through the urine producing a reddish coloration without harmful effects, a fact that the users should be alerted to.

The necrotic tissues are removed, and a cleaning, degreasing and drying with petroleum ether is carried out.

The container is first shaken and it is applied on the injured surface with a clean metal spatula or by spray.

It can be applied to the eyes, ear and mucosas without the use of cleaning solvent and without risks.

Once applied to the surface of the living tissue, physicochemical reactions occur by enzymatic catalysis, generating a semicompact web by ionic and covalent bonds between its reactive groups.

This web adheres to the damaged tissues via exposed groups of said tissues.

The diagram below is intended to show the most probable structure.

d-Panthenol oxidizes enzymatically to form pantothenic acid.

This acid can react so as to form part of the coenzyme A or to bring about its bonding to the web mentioned in the scheme.

The sulfonyl groups, the carboxyl groups, the primary or secondary hydroxyl phenol or alcohol groups can react with exposed reactive groups of proteins or amino sugars allowing the attachment of the scab to the damaged skin which presents them at a high concentration.

The forces that confer strength to the structure are intramolecular or intermolecular and of ionic or covalent type.

The bonds of the web which presents numerous crossovers are stabilized between the various layers by hydrophobic interactions, by permanent or induced dipole-dipole hydrogen bonding, and other bonds.

This produces primary, secondary, tertiary and quaternary conformations that confer three dimensionality to the structure.

The scope of the invention and the way of producing it in injured tissues (Drawing 1) having been described and specified, the declaration is made to claim as exclusive right and property:

1.—Self-fixing semipermeable porous artificial scab for protecting eroded or injured surfaces, which also allows the growth of new tissues under its protective covering. (SEE DRAWING 2).

It is a composition characterized by the formation of a semipermeable porous artificial scab which is a web produced by the physical reactions between the mentioned compounds at the site of the application, catalyzed by the enzymes present at the injured site.

It would act like a cultured skin but is a chemical polymer in as much as a semipermeable porous artificial scab having these “chemical web” characteristics does not currently exist.

It also prevents the loss of water and electrolytes plasma and proteins.

The five substances described are characterized in that they are applied to a lesion (type A-AB burn or wound) react physicochemically by catalytic action of the damaged tissues thus generating a protective layer which prevents infections and the loss of plasma and proteins.

The new tissues do not lose their original structures due to the protection provided by this layer.

### 5. DESCRIPTION OF THE DRAWINGS

### Drawing 1

Drawing 1 is intended to show a first-degree burn of the skin.

### Drawing 2

This drawing is intended to show the description of the physicochemical reactions that produce a semicompact web by ionic and covalent bonds between its reactive groups; between the different components of the formula one can see peptide bonds, hydrophobic interactions, polar interactions, permanent or induced dipole-dipole bonds, hydrogen bonds, amide, ester, acid base bonds between the various components; this generates primary, secondary, tertiary and quaternary conformations which confer three dimensionality to the structure.

### Drawing 3

Diagram of most likely chemical formula of semipermeable porous artificial scab.

### Drawing 4

Gentian violet: Methyl violet, commonly also called crystal violet or gentian violet, is the name given to a group of chemical compounds used as pH indicators and dyes. Gentian violet of (crystalline violet, methyl violet 10B, hexamethyl pararosaniline chloride) is an antifungal agent.

### Drawing 5

Dexamethasone: Is a fluorinated corticoid having a long duration of action, a high anti-inflammatory and immunosuppressant power, and a low mineralocorticoid activity. It inhibits the synthesis of prostaglandins and leukotrienes, mediator substances in the vascular and cellular processes of inflammation and immunological response.

### Drawing 6

Gentamycin: gentamycin is a broad-spectrum aminoglucoside antibiotic with bactericidal action. Mechanism of action: The aminoglucosides are transported actively through the bacterial membrane, they bind irreversibly to one or more specific receptor proteins of the subunit 30S of the bacterial ribosomes and interfere with the initiation complex between mRNA (messenger RNA) and the subunit 30S. The DNA can be read incorrectly, which gives rise to the production of nonfunctional proteins; the polyribosomes separate and are not capable of synthesizing proteins. This gives rise to an accelerated transport of aminoglucosides, as a result of which there is an increase in the rupture of the cytoplasmic membranes of the bacteria and consequent cell death.

### Drawing 7

Panthenol: Panthenol, also referred to as D-pantothenyl alcohol or pantothenol, is a colorless, highly viscous and relatively sticky liquid. It is easily soluble in water and in alcohol) (96°). Panthenol dissolves without difficulty in the formulation of hair solutions, provided they are aqueous, hydroalcoholic or hydropropylene-alcoholic solutions.

### Drawing 8

Azosulfamide: The sulfamides are basically derivatives of sulfanilamide which is a structural analog of PABA in which the carboxyl group is replaced by a sulfonyl group (SO2NH2). Thus, the sulfas act as competitive antagonists of PABA, preventing the synthesis of dihydrofolate; in the absence of that metabolite, the synthesis of nucleic acids becomes impossible and as a result bacterial growth is inhibited (bacteriostatic effect).

1. A fluid that forms an artificial scab, comprising:
1-5 weight % of azosulfamide;
0.004-0.007 weight % of gentian violet;
0.001-0.003 weight % of dexamethasone;
0.5-2 weight % of panthenol; and
0.03-0.06 weight % of gentamycin, wherein the azosulfamide, gentian violet, dexamethasone, panthenol, and gentamycin are mixed together to form an aqueous thixotropic fluid and the aqueous fluid forms an artificial scab when applied to damaged human tissue.

2. The fluid that forms an artificial scab of claim 1, further comprising a three dimensional web formed by a reaction after the application of the aqueous fluid to the damaged human tissue.

3. The fluid that forms an artificial scab of claim 1, wherein the web comprises a plurality of ionic and covalent bonds generated by the reaction after the application of the aqueous fluid to the damaged human tissue.

4. The fluid that forms an artificial scab of claim 1, wherein the artificial scab is three dimensional.

5. The fluid that forms an artificial scab of claim 1, the artificial scab having the chemical formula:

[symbol]

6. A method of forming a self-fixing semi-permeable porous artificial scab to protect damaged tissue of a patient, comprising:
mixing 1-5 weight % of azosulfamide, 0.004-0.007 weight % of gentian violet, 0.001-0.003 weight % of dexamethasone, 0.5-2 weight % of panthenol, and 0.03-0.06 weight % of gentamycin in a container to form an aqueous thixotropic fluid;
applying the aqueous thixotropic fluid to damaged tissue;
causing a physiochemical reaction to occur by enzymatic catalysis between the damaged tissue and aqueous thixotropic fluid; and
generating an artificial scab by ionic and covalent bonds that adheres to the damage tissue via exposed groups of the tissue.

7. The method of claim 6, further comprising forming the artificial scab such that the scab is three dimensional by the generation of a plurality of primary, secondary, tertiary, and quaternary conformations.

8. The method of claim 7, wherein the conformations are formed by one or more peptide bonds, hydrophobic interactions, polar interactions, permanent or induced dipole-dipole bonds, hydrogen bonds, and amide-, ester-, and acid-based bonds.

9. The method of claim 6, further comprising cleaning the damaged tissue before applying the aqueous thixotropic fluid to the damaged tissue.

10. The method of claim 6, further comprising storing the aqueous thixotropic fluid in a non-transparent glass container.

11. The method of claim 6, further comprising shaking the aqueous thixotropic fluid immediately before applying the aqueous thixotropic fluid to the damaged tissue.

12. The method of claim 6, wherein the aqueous thixotropic fluid self-fixes itself to the damage tissue after it is applied.
